SEQUENCE LISTING (4 sequences)

SEQ ID NO: 1 - protein, 572 aa, Homo sapiens
MECLYYFLGFLLLAARLPLDAAKRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLATTLKSYDSNTPGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPASPLRMANSALISVGCLAIFVTVISLLVYKKHKEYNPIENSPGNVVRSKGLSVFLNRAKAVFFPGNQEKDPLLKNQEFKGVS

SEQ ID NO: 2 - protein, 560 aa, Homo sapiens
MECLYYFLGFLLLAARLPLDAAKRFHDVLGNERPSAYMREHNQLNGWSSDENDWNEKLYPVWKRGDMRWKNSWKGGRVQAVLTSDSPALVGSNITFAVNLIFPRCQKEDANGNIVYEKNCRNEAGLSADPYVYNWTAWSEDSDGENGTGQSHHNVFPDGKPFPHHPGWRRWNFIYVFHTLGQYFQKLGRCSVRVSVNTANVTLGPQLMEVTVYRRHGRAYVPIAQVKDVYVVTDQIPVFVTMFQKNDRNSSDETFLKDLPIMFDVLIHDPSHFLNYSTINYKWSFGDNTGLFVSTNHTVNHTYVLNGTFSLNLTVKAAAPGPCPPPPPPPRPSKPTPSLGPAGDNPLELSRIPDENCQINRYGHFQATITIVEGILEVNIIQMTDVLMPVPWPESSLIDFVVTCQGSIPTEVCTIISDPTCEITQNTVCSPVDVDEMCLLTVRRTFNGSGTYCVNLTLGDDTSLALTSTLISVPDRDPASPLRMANSALISVGCLAIFVTVISLLVYKKHKEYNPIENSPGNVVRSKGLSVFLNRAKAVFFPGNQEKDPLLKNQEFKGVS

SEQ ID NO: 3 - protein, 9 aa, Homo sapiens
AYVPIAQVK

SEQ ID NO: 4 - protein, 19 aa, Homo sapiens
DVYVVTDQIPVFVTMFQKN

(19) United States
(12) Patent Application Publication
(10) Pub. No.: US 2017/0030926 A1
(43) Pub. Date: Feb. 2, 2017

(54) MARKER OF NEUROPATHIC GAUCHER'S DISEASE AND METHODS OF USE THEREOF

(71) Applicant: Yeda Research and Development Co. Ltd., Rehovot (IL)

(72) Inventors: Anthony FUTERMAN, Rehovot (IL); Hila Zigdon, Rehovot (IL)

(21) Appl. No.: 15/234,364

(22) Filed: Aug. 11, 2016

Related U.S. Application Data
(63) Continuation of application No. PCT/IL2015/050150, filed on Feb. 10, 2015.
(60) Provisional application No. 62/060,605, filed on Oct. 7, 2014.
(60) Provisional application No. 62/030,627, filed on Jul. 30, 2014.
(60) Provisional application No. 61/938,164, filed on Feb. 11, 2014.

(51) Int. Cl. G01N 33/68

(52) U.S. Cl. CPC G01N 33/6893; G01N 2333/705; G01N 2405/08; G01N 2800/044; G01N 2800/52; G01N 2800/56

(57) ABSTRACT

A biomarker of the neuronopathic types of Gaucher's disease (nGD) is provided, and use thereof for assisting the diagnosis of this form of the disease and its severity. In particular, use of the level of trans-membrane glycoprotein non-metastatic B (GPNMB) or a fragment thereof in the cerebrospinal fluid (CSF) as a diagnostic marker of nGD is provided. Further provided are methods for selecting drugs and assessing the efficacy of drugs and therapies for treating nGD.

### RELATED APPLICATIONS

This application is a Continuation of PCT Patent Application No. PCT/IL2015/050150 having International filing date of Feb. 10, 2015, which claims the benefit of priority under 35 USC 119(e) of U.S. Provisional Patent Application Nos. 62/060,605 filed on Oct. 7, 2014, 62/030,627 filed on Jul. 30, 2014 and 61/938,164 filed on Feb. 11, 2014, the contents of which are incorporated herein by reference in their entirety.

### SEQUENCE LISTING STATEMENT

The ASCII file, entitled 66946SequenceListing.txt, created on Aug. 11, 2016, comprising 10,492 bytes, submitted concurrently with the filing of this application in incorporated herein by reference.

### FIELD OF THE INVENTION

The present invention provides a biomarker for the neuronopathic types of Gaucher's disease (nGD) and use thereof for diagnosing this form of the disease and its severity, and further for selecting drugs and assessing the efficacy of drugs and therapies for treating nGD.

### BACKGROUND OF THE INVENTION

Neurodegenerative diseases are medical conditions characterized by progressive nervous system dysfunction. Neurodegenerative diseases, particularly late onset brain disorders diseases, affect an increasing number of individuals in the aging society of developed countries. Alzheimer's disease and Parkinson disease are the most prevalent diseases, but Creutzfeldt-Jacob disease (CJD), and other diseases are also increasingly diagnosed. Subjects suffering from multiple sclerosis (MS) also develop over the years features of neurodegenerative conditions.

Lysosomal Storage Disorders (LSDs) are inherited disorders caused by a defect in lysosomal function that results in accumulation of substances within the lysosome of cells. This defect is a consequence of deficiency of specific enzymes that are normally required for the breakdown of certain complex carbohydrates, and typically a defect in a single enzyme leads to the symptoms of a certain disease. Nearly 50 types and subtypes of LSDs have been identified and taken together they are estimated to affect about 1 in 7,700 births.

Gaucher disease (GD) is the most common Lysosomal Storage Disorder. This disease is caused by mutations in the Gbal gene encoding the lysosomal hydrolase, glucocerebrosidase (GlcCerase, GCase; EC 3.2.1.45), which results in accumulation of glucosylceramide (GlcCer). Patients with GD are usually classified into three types, based on the presence or absence of neurological manifestations and their rate of progression. Type 1 (or non-neuropathic type) is the most common form of the disease, occurring in approximately 1 in 50,000 live births. Type 1 patients exhibit a broad spectrum of severity, and some can remain asymptomatic throughout life. Type 2 (acute infantile) and Type 3 (juvenile or early adult onset) forms comprise only 4% of GD patients. Collectively, Type 2 and 3 are referred to as neuropathic (also known as neuronopathic) GD (nGD) since those types display Central Nervous System (CNS) involvement in addition to systemic disease. The main sign is severe difficulty (in Type 3), or a total inability (in Type 2) to generate saccades (ocular motor apraxia).

Patients with Type 2 GD usually die before 3 years of age. Type 2 patients fail to thrive, and display severe and rapidly progressive brainstem degeneration. The most frequent initial clinical signs are hyperextension of the neck, swallowing impairment and strabismus. The most common cause of death is prolonged spontaneous apnea which occurs with increased frequency in the later stages of the disease.

Type 3 patients present similar signs to Type 2 patients but with a later onset and decreased severity, and these patients usually survive until adolescence or adulthood. Eye movement abnormalities are common in nGD and their detection is diagnostic of this disorder. In Type 3 nGD, oculomotor signs may precede the appearance of overt neurological signs by many years. Auditory brainstem response (ABR) abnormalities are also an early neurological sign in nGD. These symptoms may be isolated, or appear together with developmental delay and seizures.

Current treatment of GD is Enzyme Replacement Therapy (ERT) or Bone Marrow Transplantation (BMT). Specifically, ERT provides good relief of symptoms, but this treatment is lifelong and highly expensive. In addition, ERT does not ameliorate the damage to the CNS that exists in Type 2/3 patients because the recombinant enzyme used in ERT does not cross the Blood Brain barrier (BBB). BMT is specifically described for Type 1 Gaucher patients for whom ERT is not an option.

Another strategy for treating Type 1 GD is substrate reduction therapy, employing means for inhibition of glucosylceramide biosynthesis which may improve the clinical course of the disease. This strategy also is not applicable for Type 2 and 3.

Yet another possible strategy to treat GD is gene therapy mediated by adenovirus and lentivirus vectors, although significant hurdles still exist with the implementation of gene therapy as a practical and safe therapeutic strategy.

Very little is known about the neuropathological events that cause neurological abnormalities associated with nGD. Previous systematic neuropathological analysis of autopsy human GD brains highlighted specific patterns of astrogliosis and neuron loss, in addition to non-specific gray and white matter gliosis (Wong et al., Mol. Genet. Metab., 82:192-207, 2004). A recent study conducted by some of the inventors of the present invention elucidated the onset and progression of various neuropathological changes (including microglial activation, astrogliosis and neuron loss) in a mouse model of nGD, and documented the brain areas that are first affected during the course of the disease. In addition, it was established that microglial activation and astrogliosis are spatially and temporally correlated with selective neuron loss (Farfel-Becker et al., Hum. Mol. Genet., 20:1375-1386, 2011). Neuroinflammation may be also involved in the pathogenesis of neuronopathic Gaucher's disease. A cytotoxic role has been suggested for activated microglia in neuronopathic Gaucher's disease (Vitner, Brain, 135(Pt 6):1724-35, 2012. The involvement of cathepsins in the neuropathology of neuronal forms of GD was also suggested (Vitner et al., Hum. Mol. Genet., 19:3583-3590, 2010).

Recently, some of the inventors of the present invention described that modulating the receptor-interacting protein kinase 3 (RIP3) pathway improved neurological and visceral disease in a mouse model of GD. It was demonstrated that Rip3 deficiency improved the clinical course of GD mice with increased survival, motor coordination and salutary effects on cerebral as well as hepatic injury (Vinter et al., Nat Med., 20(2):204-208, 2014).

Glycoprotein non-metastatic B (GPNMB) is a protein that was identified and described by Weterman et al., (Int J Cancer, 60:73-81, 1995) as differentially expressed in low-metastatic human melanoma cancer cell lines and xenografts, compared to a more aggressive melanoma cell line. In humans the protein is encoded by the GPNMB gene, the mouse and rat orthologues known as DC-HIL and Osteoactivin (OA), respectively. Two transcript variants encoding 560 and 572 amino acid isoforms have been characterized for this gene in humans. GPNMB is a type I transmembrane glycoprotein which shows homology to the pmel17 precursor, a melanocyte-specific protein. GPNMB has been reported to be expressed in various cell types, including melanocytes, osteoclasts, macrophages, neurons and astrocytes (Tanaka et al., Sci Rep, 2:573, 2012; Ripoll et al., J. Immunol., 178:6557-6566, 2007). Elevated levels of GPNMB were detected in the past in some lysosomal storage disorders: altered GPNMB expression was found in liver of Niemann Pick type C mice and in the brain of mucopolysaccharidosis (MPS) VII but not in MPS I or MPS IIIb mice (Cluzeau et al., Hum Mol Genet, 21:3632-3646, 2012; Parente et al., PLoS ONE, 7:e32419, 2012). Elevated expression of GPNMB was also detected in the brain of Tay-Sachs and Sandhoff patients (Cluzeau et al., 2012 ibid; Myerowitz R. et al., 2004. Mol Genet Metab 83:288-296). Urine levels of GPNMB were found to be efficient as a marker of kidney disease progression (Patel-Chamberlin M et al. 2011. Kidney Int 79:1138-1148).

GPNMB has been proposed as a diagnostic marker for several cancerous diseases. For example, International Application Publication No. WO 2008/133641 discloses antibodies with specificity to GPNMB, particularly fully human monoclonal antibodies that specifically bind to GPNMB, and uses thereof for detecting various types of cancers. The invention also provides biomarkers for evaluating the effects of therapeutic methods and uses of the antibodies with specificity to GPNMB.

U.S. Pat. No. 8,703,433 discloses the use of GPNMB as a marker for detecting amyotrophic lateral sclerosis (ALS).

U.S. Patent Application Publication No. 2014/0031411 discloses use of the GPNMB as a genetic marker in the incidence of cardiovascular conditions and cardiac diseases, such as complications derived from myocardial infarction. GPNMB is disclosed as providing a valuable tool both for diagnostic as well as therapeutic approaches, in order to treat or prevent cardiovascular conditions and cardiac diseases, in particular complications derived from myocardial infarction.

However, as of today, there is no adequate method for the early diagnosis of the neuronopathic types of Gaucher disease, and thus the therapeutic intervention is delayed. In addition, in the absence of a biomarker for this severe type of the disease there are no available assays for testing whether treatment is successful, as well as for searching candidate drugs designed to aid patient affected with neuronopathic GD (nGD).

There is an unmet need for and it would be highly advantageous to have assays based on a specific biomarker for the diagnosis and monitoring of nGD, as well as means to screen for and assess the efficacy of new therapies for nGD.

### SUMMARY OF THE INVENTION

The present invention provides according to some aspects additional means for diagnosing the more severe types of Gaucher disease, Type 2 and Type 3 collectively referred to as neuronopathic GD, based on the level of trans-membrane glycoprotein non-metastatic B (GPNMB) in the cerebrospinal fluid (CSF). The present invention further provides methods for prognosing the disease, monitoring response to treatment, and screening for new drugs and assessing their efficacy in treating nGD.

The teachings of the present invention answer the current unfulfilled need for a biomarker of nGD, which is useful in diagnosing and monitoring the progression of nGD. The diagnosis of nGD, particularly at an early stage, can facilitate a more effective treatment to this severe form of the disease. Monitoring progression is important, inter alia, for evaluating the effect of therapy. Furthermore, the biomarker of the invention is useful in the search for and development of new drugs effective in treating nGD.

The present invention is based in part on the unexpected discovery that the trans-membrane glycoprotein non-metastatic B (GPNMB) is present in the cerebrospinal fluid (CSF) of subjects affected with neuronopathic Type 3 GD at significant higher levels compared to its level in healthy subjects. Furthermore, the level of GPNMB was found to be positively correlated with the severity of the disease. Thus, assessing the level of GPNMB enables not only the diagnosis of the disease, but also to assess the disease progress or remission in the regular course of the disease or after administration of a drug.

According to one aspect, the present invention provides a method for assessing the responsiveness of a subject diagnosed with neuronopathic Gaucher's disease (nGD) to a treatment, the method comprising:
(a) measuring a level of GPNMB or a fragment thereof in a first cerebrospinal fluid (CSF) sample obtained from the subject;
(b) administering the treatment to said subject;
(c) measuring a level of GPNMB or the fragment thereof in a second CSF sample obtained from said subject at a selected time period after the treatment was administered; and
(d) calculating a ratio of the level of GPNMB or the fragment thereof in the first CSF sample to the level of GPNMB or the fragment thereof in the second CSF sample;

wherein a ratio of greater than 1 identifies said subject as responsive to said treatment.

According to certain embodiments, GPNMB used as a biomarker in the methods of the present invention is any one of the known protein isoforms or a fragment thereof. According to certain embodiments, GPNMB comprises the amino acid sequence set forth in SEQ ID NO: 1 (UniProtKB/Swiss-Prot Q14956.2). According to other embodiments, GPNMB comprises the amino acid sequence set forth in SEQ ID NO: 2 (NCBI: NP 002501). In some embodiments, GPNMB has an amino acid sequence selected from the group consisting of SEQ ID NO: 1 and SEQ ID NO: 2.

As used herein, a “fragment” of GPNMB typically refers to at least 10 successive amino acids from the sequence of the particular isoform of GPNMB. In some embodiments, a fragment comprises at least 15 successive amino acids from the sequence of the particular isoform of GPNMB. According to some embodiments, the protein fragment is derived from the extracellular domain of the protein. According to some embodiments, the protein fragment is derived from positions 1-499 of SEQ ID NO: 1. According to additional embodiments, the protein fragment is derived from positions 1-490 of SEQ ID NO: 2. According to certain exemplary embodiments, the fragment comprises the amino acid sequence AYVPIAQVK (SEQ ID NO: 3). According to other exemplary embodiments, the fragment comprises the amino acid sequence DVYVVTDQIPVFVTMFQKN (SEQ ID NO: 4).

In some embodiments, measuring a level of GPNMB or a fragment thereof is performed using an immunologic technique.

In some embodiments, the immunologic technique is selected from the group consisting of fluorescence immunoassay (FIA) method, an enzyme immunoassay (EIA) method, a radioimmunoas say (RIA) method, a Western blotting method and slot blot. Each possibility represents a separate embodiment of the present invention.

In some embodiments, the method further comprises repeating steps (c) and (d) at least once, wherein a third or more samples are obtained in step (c) according to the number of repeats.

In some embodiments, the time period after the treatment was administered ranges between 7-360 days, for example between 7-180 days, between 7-120 days, between 7-30 days. In some embodiments, the time period after the treatment was administered ranges between 28-360 days, for example between 28-180 days, between 28-120 days. Each possibility represents a separate embodiment of the present invention.

The treatment administered to the subject can be any currently known treatment to GD, particularly to nGD, or any treatment to be developed in the future.

According to certain embodiments, the subject is human.

According to an additional aspect, the present invention provides a method for diagnosing neuronopathic Gaucher's disease (nGD) in a subject, the method comprising:
(a) receiving a cerebrospinal fluid (CSF) sample from a subject suspected of having nGD;
(b) determining a level of GPNMB or a fragment thereof in the CSF sample using a quantitative assay;
(c) comparing the level of GPNMB or the fragment thereof in the CSF sample to a reference value representing a normal level of GPNMB in the CSF; and
(d) diagnosing the subject as having nGD if the level of GPNMB or the fragment thereof in the CSF sample is above the reference value.

In some embodiments, the quantitative assay used for determining a level of GPNMB or a fragment thereof in the CSF sample is an immunoassay.

In some embodiments, the immunoassay is selected from the group consisting of fluorescence immunoassay (FIA) method, an enzyme immunoassay (EIA) method, a radioimmunoassay (RIA) method, a Western blotting method and slot blot. Each possibility represents a separate embodiment of the present invention.

In some embodiments, the method further comprises administering treatment to the subject.

In some embodiments, the method further comprises determining disease severity.

In some embodiments, disease severity is determined based on the degree of increase in the level of GPNMB or the fragment thereof measured in the CSF sample from the subject relative to the reference value representing a normal level of GPNMB in CSF. In some embodiments, determining disease severity comprises correlating the level of GPNMB or a fragment thereof measured in a CSF sample from the subject to a set of predetermined reference values, wherein each of the reference values indicates a degree of severity.

In some embodiments, determining disease severity comprises providing a plurality of reference values, each representing a level of GPNMB in the CSF that is characteristic of a degree of disease severity; comparing the level of GPNMB or the fragment thereof measured in the CSF sample from the subject to each reference value; and determining the degree of disease severity in the subject based on the reference value which correlates best with the level of GPNMB or the fragment thereof in the CSF sample from the subject.

In some embodiments, each reference value represents a range of levels of GPNMB in the CSF that is characteristic of a degree of disease severity.

According to another aspect, the present invention provides a method for monitoring the progression of neuronopathic Gaucher disease (nGD) in a subject diagnosed with nGD, based on monitoring a change in the levels of GPNMB in the CSF of the subject. An increase in the level of GPNMB in the CSF is indicative of disease progression and worsening. Similarly, a decrease in the level of GPNMB in the CSF is indicative of improvement.

In some embodiments, monitoring comprises:
(a) measuring a first level of GPNMB or a fragment thereof in a first CSF sample taken from a subject diagnosed with nGD;
(b) measuring a second level of GPNMB or the fragment thereof in a second CSF sample taken from the subject after a predetermined period of time;
(c) comparing the first and second levels of GPNMB or the fragment thereof; and
(d) determining disease progression if the second level of GPNMB or the fragment thereof is significantly increased compared to the first level of GPNMB or the fragment thereof.

In some embodiments, the first level of GPNMB or the fragment thereof is measured before treatment and the second level of GPNMB or the fragment thereof is measured after treatment. In some embodiments, the method comprises determining improvement of the disease if the second level of GPNMB or the fragment thereof measured after treatment is significantly reduced compared to the first level of GPNMB or the fragment thereof measured before treatment.

In some embodiments, the predetermined period of time between a first and second sampling of CSF ranges between 7-360 days, for example between 7-180 days, between 7-120 days, between 7-30 days. In some embodiments, the predetermined period of time between a first and second sampling of CSF ranges between 28-360 days, for example between 28-180 days, between 28-120 days. Each possibility represents a separate embodiment of the present invention.

According to a further aspect, the present invention provides a method of screening for a compound or a therapy for treating neuronopathic Gaucher's disease (nGD), comprising:
(a) measuring a level of GPNMB or a fragment thereof in a first cerebrospinal fluid (CSF) sample obtained from at least one test non-human mammal affected with nGD;
(b) administering to the at least one test non-human mammal at least one candidate compound or a candidate therapy for treating nGD;
(c) measuring a level of GPNMB or the fragment thereof in a second CSF sample obtained from said at least one test non-human mammal at a selected time point after the candidate compound or therapy was administered; and
(d) calculating a ratio of the level of GPNMB or the fragment thereof in the first CSF sample to the level of GPNMB or the fragment thereof in the second CSF sample;

wherein a ratio of greater than 1 identifies the at least one candidate compound or therapy as effective in treating nGD.

According to some embodiments, the method comprises the steps of:
(a) obtaining at least two test non-human mammals affected with nGD;
(b) administering at least one candidate compound or a candidate therapy for treating nGD to part of the test non-human mammals, to obtain at least one treated test mammal and at least one control test mammal;
(c) measuring a level of GPNMB or the fragment thereof in a CSF sample obtained from the at least one treated test mammal and the at the least one control test mammal at a selected time period after the candidate compound or candidate therapy was administered; and
(d) calculating a ratio of the level of GPNMB or the fragment thereof in the CSF sample obtained from said at least one control test mammal to the level of GPNMB or the fragment thereof in the sample obtained from said at least one treated test mammal;

wherein a ratio of greater than 1 identifies the at least one candidate compound or therapy as effective in treating nGD.

According to certain embodiments, the method comprises repeating steps (c) and (d) at least once, wherein a third or more samples are obtained in step (c) according to the number of repeats.

Setting the time point after which the level of GPNMB or the fragment thereof is examined in the second or further CSF samples can be determined according to the type of the test animal, the amount of candidate compound administered, the number of repeats and the like.

According to some embodiments, the second sample is obtained at a time point between about 1-120 days after administration of the at least one test compound or therapy.

According to certain embodiments, the test non-human animal is selected from the group consisting of mice and sheep. Each possibility represents a separate embodiment of the present invention.

According to some embodiments, the test non-human animal is a mouse. According to certain exemplary embodiments, the mouse is genetically induced to express nGD symptoms. According to other exemplary embodiments, the mouse is chemically induced to express nGD symptoms.

The candidate compound can be administered by any route as suitable for the specific compound and as is known to a person skilled in the art. According to some embodiments, the at least one candidate compound is administered in a form selected from the group consisting of intravenously (i.v.), orally also known as per os (p.o.), intraperitoneally (i.p), intranasally, topically, by inhalation, and via eye drops. Each possibility represents a separate embodiment of the present invention.

According to yet additional aspect, the present invention provides a kit for diagnosing or assessing the severity of neuronopathic Gaucher's disease (nGD), comprising at least one reagent for detecting a level of GPNMB or a fragment thereof in a cerebrospinal fluid (CSF) sample obtained from a subject suspected to have or having nGD, and instructional material directing the correlation between said detected level of GPNMB or a fragment thereof and a predetermined reference control level of GPNMB or predetermined set of reference levels each of which indicating a degree of disease severity.

In some embodiments, the predetermined reference control level of GPNMB is a threshold level above which the subject is diagnosed as having nGD.

In some embodiments, the predetermined set of reference levels is a set of ranges of reference levels, each range being characteristic of a degree of disease severity.

According to certain embodiments, the reagent for detecting GPNMB or a fragment thereof is an antibody specifically recognizing GPNMB or the fragment thereof. According to some embodiments, the kit further comprises means for performing the detection. According to certain embodiments, the means for performing the detection are selected from the group consisting of reagents for performing an ELISA, an RIA, a slot blot, an immunohistochemical assay, FACS, in vivo imaging, a radio-imaging assay, or a Western blot.

Other objects, features and advantages of the present invention will become clear from the following description and drawings.

### BRIEF DESCRIPTION OF THE SEVERAL VIEWS OF THE DRAWINGS

FIGS. 1A-B show hyperphosphorylation of Tau in nGD samples. FIG. 1A: Western blot of Tau and P-Tau (using two different anti-P-Tau antibodies) in brains of 21 day-old Gba^{flox/flox}; nestin-Cre mice; FIG. 1B; P-Tau in the brain of one Type 2 GD patient. Results are from 3 repeats of the experiment. GAPDH was used as a loading control. A molecular weight marker is shown (Mr=55 kDa).

FIGS. 2A-D demonstrate the elevation of GPNMB levels in CSF and brain samples of Type 2 and Type 3 Gaucher patients. FIG. 2A: levels of GPNMB determined by LC-MS/MS analysis in CSF samples of four Type 3 GD patients. Results are means±SEM (n=4); ** p<0.01. FIG. 2B: Levels of GPNMB in CSF samples of four Type 3 GD patients determined by ELISA. Results are means±SEM (n=4). **P<0.01. FIG. 2C: Western blot analysis of GPNMB levels in CSF samples of control and Type 3 Gaucher patients. Results are from 3 repeats of the experiment. FIG. 2D: Levels of GPNMB in brain tissue of control and nGD patients determined by ELISA (n=3 for control, n=6 for nGD (Type 2 and Type 3 patients). Results are means±SEM, ** p<0.01.

FIGS. 3A-B demonstrate the elevation in GPNMB levels in brain and serum samples of Gba^{flox/flox}; nestin-Cre using ELISA. FIG. 3A: Levels of GPNMB in brain (n=3) at different days post-natal (p). Results are means ±SEM *P<0.05, ** P<0.01. FIG. 3B: Levels of GPNMB in serum of 21-day old mice (n=4, n=5).

FIGS. 4A-D show GPNMB levels in CSF and brain samples of CBE treated mice. FIG. 4A: Experimental regime. FIG. 4B: mice weight as an indication for disease severity. FIGS. 4C-D: GPNMB levels in CSF samples (FIG. 4C) and in the brain samples (FIG. 4D) measured by ELISA. Results are means±SEM. * p<0.06, ** p<0.01.

### DESCRIPTION OF THE SPECIFIC EMBODIMENTS OF THE INVENTION

The present invention is directed, inter alia, to detection and monitoring of deterioration in neurodegenerative diseases. In particular embodiments, the present invention is directed to additional means and methods for diagnosing and monitoring subjects suspected to have or having the neuronopathic form of Gaucher's disease. The present invention is also directed to means and methods for assessing the disease severity, and further for selecting treatment compounds and/or therapies for treating said subjects. The means and methods of the present invention are based on measuring the level of GPNMB in a CSF sample from a subject. In some embodiments, the means and methods of the present invention are based on measuring the level of a fragment of GPNMB in a CSF sample of a subject. In some embodiments, the fragment is derived from the extracellular domain of GPNMB. In some embodiment, the fragment is the extracellular domain of GPNMB. In some embodiment, the fragment is derived from, or directly corresponds to, positions 1-499 of SEQ ID NO: 1. According to additional embodiments, the fragment is derived from, or directly corresponds to, positions 1-490 of SEQ ID NO: 2.

As of today, there are no available biomarkers for the more severe, neuronopathic form of Gaucher's disease. This deficiency limits the ability to search for candidate agents that may be useful in treating the disease, as there is no reliable means for assessing the efficacy of the candidate agents. Furthermore, subjects are diagnosed to have Gaucher's disease but not its specific type, and are thus not receiving the optimal treatment.

The present invention now shows that the level of GPNMB within a body fluid sample, particularly CSF, is positively correlated with the presence and severity of nGD.

Definitions

The term “neuronopathic Gaucher's disease or “nGD” refers to the Type 2 and Type 3 forms of Gaucher' s disease as are known in the art and as described in the background section hereinabove.

As used herein the term “level” typically means “amount” or “concentration”. However, the term “level” is also used in the case of expressing whether a molecule to be detected can be detected or not (that is, presence or absence of detectable existence), according to the custom and technical common knowledge.

As used herein, “determination of a level”, “determining a level” or “measuring a level” typically refer to calculation of amount or concentration of a particular substance, or to quantifying an intensity of a signal from a probe that represents the amount or concentration of a particular substance. For example, determining a level may include quantifying fluorescence or radioactivity emission from a probe.

The term “signal” is used generally in reference to any detectable process that indicates that a reaction has occurred, for example, binding of an antibody to an antigen. It is contemplated that signals in the form of radioactivity, fluorometry or colorimetry will all find use with the present invention. In some currently preferred embodiments, the signal is assessed quantitatively.

As used herein, “elevation”, “increase” and the like, of a measured value as compared to another value, typically refer to statistically significant differences.

As used herein, a “subject” commonly refers to mammalian subject. A mammalian subject may be human or non-human, preferably human.

The terms “affected with” or “having”, with respect to a disease, are used herein interchangeably and refer to subjects that are carriers of the disease, regardless of the degree of symptom manifestation. The affected subjects can be at any disease phase, including, but not limited to, before burst, at burst, during a continuous course of the disease and after remission.

Accordingly, “a subject affected with neuronopathic Gaucher's” refers to a subject with nGD showing symptoms, a subject with nGD being subject in remission, a subject with nGD with manifested symptoms and a subject susceptible to nGD. Each possibility is a separate embodiment of the invention. According to certain embodiments, “susceptible to nGD” refers to a subject having genetic makeup which enhances the chance of the subject to show symptoms of GD.

As used herein, the term “a subject suspected to have nGD” refers to a subject susceptible to nGD and/or a subject manifesting symptoms suspected to indicate nGD.

As used herein, a “subject diagnosed with nGD” refers to a subject that has been determined to have nGD, by methods known in the art or by the methods of the present invention. In some embodiments, the subject has been shown to have genetic mutations associated with the disease, and characteristic symptoms, including for example, eye movement abnormalities and auditory brainstem response (ABR) abnormalities.

The phrase “diagnosing and/or assessing a severity” as used herein refers to determining presence or absence of a disease, classifying a disease severity or symptom, monitoring disease progression, forecasting an outcome of a disease and/or prospects of recovery.

The term “treating” refers to inhibiting or arresting the development of a disease and/or causing the reduction, remission, or regression of a disease.

The present invention now discloses GPNMB or a fragment thereof as a biomarker that is useful, inter alia, in assessing the results of a treatment of nGD.

GPNMB is a transmembrane glycoprotein showing homology to melanocyte specific protein Pmel17. Two transcription variants for GPNMB are known: an isoform made of 572 amino acids (SEQ ID NO: 1 UniProtKB/Swiss-Prot Q14956.2) and an additional isoform made of 560 amino acids (SEQ ID NO: 2, NCBI: NM 002510). In the first isoform (SEQ ID NO: 1), the non-cytosolic (extracellular) domain corresponds to positions 1-499 of the sequence. In the second isoform (SEQ ID NO: 2), the non-cytosolic (extracellular) domain corresponds to positions 1-490. It has been reported that GPNMB is highly expressed in specific kinds of cancers (including melanoma, glioma, and breast cancer), and GPNMB has been suggested as a target for antibody therapy against melanoma and breast cancer.

The present invention now shows that the level of GPNMB within a sample of CSF is positively correlated with the presence and severity of nGD.

High levels of CHIT1 were also detected in the CSF of GD Type 3 patients, as exemplified hereinbelow. It was reported in the past, that there is no correlation between chitotriosidase levels and the severity of clinical manifestations (Hollak et al., J Clin Invest, 93:1288-1292, 1994). The present invention shows that similar to GPNMB, there is a connection between CHIT1 levels and disease severity yet it is less correlative. CHIT1 cannot be used to evaluate disease severity in mice models, probably since stimulation of rat and mouse macrophages do not cause increase in CHIT1 levels (Korolenko et al. Bull Exp Biol Med, 130:948-950, 2000). Thus, as oppose to CHIT1, GPNMB is useful in the preclinical step of drug development and can be further used as a biomarker to evaluate new drug efficacy in animals.

According to an aspect of the present invention, there is provided herein a method for assessing the responsiveness of a subject diagnosed with neuronopathic Gaucher's disease (nGD) to a treatment, the method comprising:
(a) measuring a level of GPNMB or a fragment thereof in a first cerebrospinal fluid (CSF) sample obtained from the subject;
(b) administering the treatment to said subject;
(c) measuring a level of GPNMB or the fragment thereof in a second CSF sample obtained from said subject at a selected time period after the treatment was administered; and
(d) calculating a ratio of the level of GPNMB or the fragment thereof in the first CSF sample to the level of GPNMB or the fragment thereof in the second CSF sample;

wherein a ratio of greater than 1 identifies said subject as responsive to said treatment.

In some embodiments, the method further comprises repeating steps (c) and (d) at least once, wherein a third or more samples are obtained in step (c) according to the number of repeats.

According to a further aspect, there is provided herein a method for identifying a subject as having nGD, the method comprising:
(a) receiving a cerebrospinal fluid (CSF) sample from a subject suspected of having nGD;
(b) determining a level of GPNMB or a fragment thereof in the CSF sample using a quantitative assay;
(c) comparing the level of GPNMB or the fragment thereof in the CSF sample to a predetermined reference value; and
(d) diagnosing the subject as having nGD if the level of GPNMB or the fragment thereof in the CSF sample is elevated compared to the predetermined reference value.

In some embodiments, the reference value represents a normal level of GPNMB or a fragment thereof in the CSF. A normal level is typically the level of GPNMB or the fragment thereof determined in CSF samples from control subjects not afflicted with GD. In some embodiments, the reference value is a threshold value, which differentiates between subjects having nGD and healthy subjects, not affected with nGD. The threshold value is therefore a value above which a subject is diagnosed with nGD. Values lower than the threshold value are indicative that the subject is not affected with nGD. The reference value is a statistically significant value. According to some embodiments, determining the reference value includes measuring the level of GPNMB or a fragment thereof in a large population of healthy subjects not affected with nGD. In some embodiments, the method comprises determining a threshold value for GPNMB level in the CSF above which a subject is diagnosed with nGD.

According to yet a further aspect, there is provided herein a method for identifying a subject to be diagnosed with and treated for neuronopathic Gaucher's disease (nGD), the method comprising:
(a) obtaining a cerebrospinal fluid (CSF) sample from a subject suspected of having nGD;
(b) determining a level of GPNMB or a fragment thereof in the CSF sample;
(c) calculating a ratio of the level of GPNMB or the fragment thereof in said CSF sample to a level of GPNMB or the fragment thereof in a CSF sample obtained from a healthy subject or to a predetermined reference value; and
(d) identifying the subject as a subject diagnosed with and to be treated for nGD when the calculated ratio is more than 1.

According to certain embodiments, the method further comprises a step of assessing the severity of nGD, the step comprising correlating the calculated ratio to a set of predetermined reference ratios, wherein each of the reference ratios indicates a degree of symptom severity.

The collection of CSF samples is performed by methods known in the art, typically by lumbar puncture, also known as spinal tap. After its collection, the CSF sample may be used with the methods of the present invention without further processing.

Any method as is known in the art for the detection of a protein in a fluid sample can be used with the teachings of the present invention. According to certain embodiments, the assay is an immunoassay.

According to certain embodiments, the methods of the present invention comprise detecting the GPNMB level in CSF samples obtained from a subject having or suspected to have nGD and in CSF samples obtained from a healthy subject. According to these embodiments, accurate quantitative determination of the level of the biomarker is not essential. In some embodiments, the level of the biomarker may be detected to a degree in which it is possible to calculate a ratio between the GPNMB levels in two samples. In addition, the detection can also be carried out so as to be able to determine whether the level of the biomarker in the examined sample exceeds the predetermined standard, reference value.

According to certain exemplary embodiments, the GPNMB level is determined using an immunologic technique. The immunologic technique enables the rapid detection with high sensitivity. Furthermore, the immunologic technique can be carried out in an easy and simple manner. In measurement by the immunologic technique, a substance having a specific binding activity with respect to the biomarker is used. As the substance, an antibody is generally used. However, the substance is not necessarily limited to the antibody, and any substances can be used as long as they have a specific binding activity with respect to the biomarker and the binding amount can be measured. Other than antibodies commercially available, newly prepared antibodies by an immunologic technique, a phage display method or a ribosome display method can be used.

In some embodiments, the methods of the present invention comprise contacting a CSF sample from a subject with a binding reagent (e.g. an antibody) that specifically recognizes and binds an epitope on GPNMB, quantifying the binding to the antibody in the CSF sample from the subject (i.e., quantifying the amount of complexes formed between the binding reagent and its antigen in the sample), and comparing said binding to a reference value representing binding between the binding reagent and its antigen determined in CSF samples from control subjects not afflicted with GD.

Detectable labels suitable for conjugation to antibodies and other binding reagents include radioisotopes, fluorescent labels, enzyme-substrate labels, chromogenic labels, chemiluminescent labels and colloidal gold particles.

Radioisotopes include for example, ³⁵ S , ¹⁴C, ¹²⁵I, ³H, ³²P and ¹³¹I. Flourescent labels include for example, fluorescent molecules such as fluorescein isothiocyanate (FITC), rhodamine, phycoerythrin (PE), phycocyanin, allophycocyanin, ortho-phthaldehyde, fluorescamine, peridinin-chlorophyll a (PerCP), Cy3 (indocarbocyanine), Cy5 (indodicarbocyanine), lanthanide phosphors, and the like.

Enzymatic labels include luciferases (e.g. firefly luciferase and bacterial luciferase), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Examples of enzyme-substrate combinations include, for example: horseradish peroxidase (HRPO) with hydrogen peroxidase as a substrate, wherein the hydrogen peroxidase oxidizes a dye precursor (e.g., orthophenylene diamine (OPD) or 3,3′, 5,5′-tetramethyl benzidine hydrochloride (TMB)); alkaline phosphatase (AP) with paranitrophenyl phosphate as chromogenic substrate; and β-D-galactosidase β-D-Gal) witha chromogenic substrate (e.g., p-nitrophenyl-β-D-galactosidase) or fluorogenic substrate (e.g., 4-methylumbelliferyl-β-D-galactosidase).

Detection of bound, labeled antibody can be carried out by standard colorimetric, radioactive, photometric and/or fluorescent detection means.

Examples of the measurement method include, but are not limited to, a fluorescence immunoassay (FIA) method, an enzyme immunoassay (EIA) method, a radioimmunoassay (RIA) method, a Western blotting method, dot blot, an immunohistochemical assay, Fluorescence Activated Cell Sorter (FACS), in vivo imaging and a radio-imaging assay. According to certain exemplary embodiments, the measurement method is selected from FIA method and an EIA method (including an ELISA method). With these methods, detection can be carried out with high sensitivity, rapidly and in a simple and easy manner. In the FIA method, a fluorescent labeled antibody is used, and an antigen-antibody complex (an immune complex) is detected by using fluorescence as a signal. In the EIA method, an enzyme-labeled antibody is used, and an immune complex is detected by using coloring and light emission based on the enzyme reaction as a signal.

The ELISA method has many advantages, for example, detection sensitivity is high, specificity is high, it enables quantitative measurements, the operation is simple and multiple specimens can be handled simultaneously. One example of a specific operation in which the ELISA method is used is described hereinafter. Firstly, an anti-biomarker antibody is immobilized to an insoluble support. Specifically, for example, the surface of a microplate is sensitized (coated) with an anti-biomarker monoclonal antibody. A specimen (CSF sample) is brought into contact with the thus solid-phased antibody. As a result of this operation, when an antigen (protein molecule, namely the biomarker) against the solid-phased anti-biomarker antibody is present in the specimen, an immune complex is formed. Non-specific binding components are removed by washing, followed by adding an antibody to which an enzyme is bound so as to label the immune complex. Then, the substrate of the enzyme is reacted to develop color. Thus, the immune complex is detected using an amount of color development as an indicator. Since the detail of the ELISA method is described in many text books or papers, when experiment procedures or experiment conditions of each method are set, such books or papers can be referred to. Note here that not only noncompetitive methods but also competitive methods (methods in which an antigen is added together with a specimen so as to allow them to compete with each other) may be used. A method of directly detecting the biomarker in a specimen with a labeled antibody may be employed or a sandwich method may be employed. In the sandwich method, two types of antibodies (a capturing antibody and a detecting antibody) whose epitopes are different from each other are used.

In some embodiments, the diagnostic methods of the present invention further comprise determining disease severity. In some embodiments, determining disease severity comprises comparing the level of GPNMB of the fragment thereof measured in the CSF sample from the subject to a plurality of reference values, each representing a level of GPNMB characteristic of a degree of disease severity, wherein the reference value which best approximates the level of GPNMB of the fragment thereof in the CSF sample of the subject is indicative of the degree of severity of the disease.

In some embodiments, each reference value represents a range of levels of GPNMB in the CSF that is characteristic of a particular degree of disease severity.

The present invention further provides a method for monitoring the progression of neuronopathic Gaucher disease (nGD) in a subject already diagnosed with nGD. In some embodiments, monitoring can be used to evaluate whether a particular treatment is successful.

In some embodiments, monitoring disease progression comprises determining a first level of GPNMB or a fragment thereof in a first CSF sample obtained from a subject diagnosed with nGD; and determining a second level of GPNMB or the fragment thereof in a second CSF sample obtained from the subject after a predetermined period of time; comparing the first and second levels of GPNMB or the fragment thereof; wherein a higher level of GPNMB or the fragment thereof in the second CSF sample compared to the first CSF sample is indicative of disease progression and worsening. Similarly, a lower level of GPNMB or the fragment thereof in the second CSF sample compared to the first CSF sample is indicative of improvement.

The diagnostic methods of the present invention may be combined with the known diagnostic methods for this type of disease.

According to certain aspects, the present invention provides a method for detecting and monitoring neurodegeneration in a subject, the method comprising: (a) measuring a level of GPNMB or a fragment thereof in a cerebrospinal fluid (CSF) sample from said subject; and (b) determining neurodegeneration in the subject if the measured level of GPNMB or the fragment thereof is significantly increased compared to a level of GPNMB or the fragment thereof determined in control healthy subjects.

According to additional aspects, the present invention provides a method for detecting and monitoring deterioration and progression of a neurodegenerative disease in a subject having a neurodegenerative disease, the method comprising: (a) measuring a first level of GPNMB or a fragment thereof in a first CSF sample from said subject; (b) measuring a second level of GPNMB or the fragment thereof in a second CSF sample from said subject taken after a predetermined period of time; (c) comparing the first and second levels of GPNMB or the fragment thereof; and (d) determining deterioration and progression of the neurodegenerative disease in the subject if the second level of GPNMB or the fragment thereof is significantly increased compared to first level of GPNMB or the fragment thereof.

In some embodiments, the neurodegenerative disease is other than amyotrophic lateral sclerosis (ALS).

In some embodiments, the neurodegenerative disease is a neurodegenerative disease characterized by central nervous system (CNS) deterioration/defects.

In some embodiments, the subject is having or suspected of having a neurodegenerative disease selected from the group consisting of multiple sclerosis (MS), Alzheimer disease, Parkinson disease, Huntington's disease and Creutzfeldt-Jacob disease (CJD). Each possibility represents a separate embodiment of the present invention.

In some embodiments, the neurodegenerative disease is a lysosomal storage disease that displays central nervous system (CNS) involvement/symptoms.

In some embodiments, the subject is having Type 2 or 3 Gaucher disease (GD), also termed neuronopathic GD (nGD).

According to certain additional aspects, the present invention provides a method of screening for a compound or a therapy for treating neuronopathic Gaucher's disease (nGD), comprising:
(a) measuring the level of GPNMB or a fragment thereof in a first cerebrospinal fluid (CSF) sample obtained from at least one test non-human mammal affected with nGD;
(b) administering to the at least one test non-human mammal at least one candidate compound or a candidate therapy for treating nGD;
(c) measuring the level of GPNMB or fragment thereof in a second CSF sample obtained from said at least one test non-human mammal after a selected time period after the candidate compound was administered; and
(d) calculating the ratio of the level of GPNMB or fragment thereof in the first sample to the level of GPNMB or fragment thereof in the second sample;

wherein a ratio of more than 1 identify the at least one candidate compound or therapy as effective in treating nGD.

According to certain embodiments, the method comprises repeating steps (c) and (d) at least once, wherein a third or more samples are obtained in step (c) according to the number of repeats.

According to yet additional aspects, the present invention provides a kit for diagnosing or assessing the severity of neuronopathic Gaucher's disease (nGD), comprising at least one reagent for detecting the level of GPNMB in a cerebrospinal fluid sample obtained from a subject suspected to have or having nGD and instructional material directing the correlation between said detected level of GPNMB and a predetermined reference control level of GPNMB or predetermined set of reference levels each of which indicating a degree of disease severity.

Any reagent for detecting the level of GPNMB can be used according to the teachings of the present invention, as long as it shows specific binding activity to GPNMB.

According to certain exemplary embodiments, the reagent is an antibody. According to these embodiments, the antibody can be a polyclonal antibody, an oligoclonal antibody (a mixture of several to several tens of kinds of antibodies) and a monoclonal antibody. An affinity purified antibody by antigen can be used as the polyclonal antibody or the oligoclonal antibody, in addition to an antiserum-derived IgG fraction obtained by immunizing animals. The antibody may be antibody fragment such as Fab, Fab′, F(ab′)₂, scFv, dsFv antibodies.

In some embodiments, the reagent for detecting GPNMB or a fragment thereof is immobilized to a surface.

The detection reagents are present in the kit in an amount effective to permit detection of the protein of interest.

Detection of the proteins is accomplished using any of the methods described herein or known to a skilled artisan for detecting a specific protein within a biological fluid sample. According to some embodiments, the kit further comprises means for performing the detection assay.

In some embodiment, the kit further comprises a container, a sample tube, or the like, for storing the biological sample obtained from the subject.

As used herein, an “instructional material” is typically in the form of a package insert and may include text material, a diagram or any other form of direction which dictates the use of the components of the kit.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Materials and Methods

GPNMB Quantification in CSF and Brain of nGD Patients:

Spinal fluid samples were collected for biomarker discovery from patients with Type 3 GD and age-matched control subjects. All GD patients were on long-term enzyme replacement therapy (ERT) as well as on miglustat (neither had any therapeutic effect on the brain).

Brains from Type 2 and Type 3 GD patients were obtained post-mortem with informed consent between 7 and 22 h after death. After removal, brains were frozen on dry ice. GD patients were classified before death as types 1, 2 or 3 based on the clinical course of the disease, and in most cases, mutational analysis was also performed. Control human brains were frozen within 6-26 h of death.

LC-MS/MS Analysis

Proteins were first reduced by incubation with dithiothreitol (DTT, 5 mM; Sigma) for 30 min at 60° C., and alkylated with 10 mM iodoacetamide (Sigma) in the dark for 30 min at 21° C. Proteins were then subjected to digestion with trypsin (Promega; Madison, Wis., USA) or to digestion with chymotrypsin for 6 h and then trypsin for 16 h at 37° C. The digestions were stopped by trifluroacetic acid (1%). After digestion was stopped the samples were stored in −80° C.

ULC/MS grade solvents were used for all chromatographic steps. Each sample was loaded using split-less nano-Ultra Performance Liquid Chromatography (10 kpsi nanoAcquity; Waters, Milford, Mass., USA). The mobile phase was: (A) H₂O+0.1% formic acid and (B) acetonitrile +0.1% formic acid. Desalting of the samples was performed online using a reversed-phase C18 trapping column (180 μm internal diameter, 20 mm length, 5 μm particle size; Waters). The peptides in the samples were separated using a C18 T3 HSS nano-column (75 μm internal diameter, 250 mm length, 1.8 μm particle size; Waters) at 0.25 μl/min. Peptides were eluted from the column into the mass spectrometer using the following gradient of phase B: 4% to 8% for 10 min, 8% to 20% for 80 min, 20% to 35% for 10 min, 35% to 90% for 5 min, maintained at 95% for 5 min and then back to initial conditions. The nanoUPLC was coupled online through a nanoESI emitter (10 μm tip; New Objective; Woburn, Mass., USA) to a quadrupole orbitrap mass spectrometer (Q Exactive, Thermo Scientific) using a FlexIon nanospray apparatus (Proxeon). Data was acquired in DDA mode, using a Top 12 method described by Kelstrup et al., J Proteome Res, 11(6):3487-97, 2012) Raw data was imported into the TransOmics® software (Waters) (also known as Progenesis® LC-MS). The software was used for retention time alignment and peak detection of precursor peptides. A master peak list was generated from all MS/MS events and sent for database searching using Mascot v2.4 (Matrix Sciences). Data was searched against forward and reversed human sequences of UniprotKB version 05_2012 including 125 common laboratory contaminants. Fixed modification was set to carbamidomethylation of cysteines and variable modification was set to oxidation of methionines. Search results were then imported back to TransOmics to annotate identified peaks. Identifications were filtered such that the global false discovery rate was maximum of 1%. Differential analysis was conducted by direct comparison of aligned peptide intensities across all samples. Technical replicates were averaged and a Student's t-Test, after logarithmic transformation, was used to identify significant differences across the biological replica.

Western Blot Analysis

Brain homogenates or CSF samples were electrophoresed on a 10% SDS-polyacrylamide gel and transferred to a nitrocellulose membrane. Blots were incubated with the primary anti-GPNMB antibodies (R&D systems) and anti-Albumin (Dako Cytomation), anti-Tau (cell signaling), anti-P-Tau (cell signaling) and AT8 (innogenetics) followed by a horseradish peroxidase-conjugated secondary antibody. Bound antibodies were detected using the SuperSignal West Pico chemiluminescent substrate (Thermo Scientific)

Enzyme-Linked Immunosorbent Assay

GPNMB levels were measured in CSF aspirated from Type 3 GD patients and in brain samples of Type 2 and 3 GD patients. Brain tissues were lysed in ˜6 volumes of Ripa buffer (150 mM sodium chloride, 1.0% Triton X-100, 0.5% sodium deoxycholate, 0.1% SDS, 50 mM Tris, pH 8.0) supplemented with a protease inhibitor mixture (Sigma). Following homogenization, samples were centrifuged at 4500 g, for 5 min at 4° C., and the supernatant was collected. Protein was quantified using the BCA protein assay reagent (Pierce Chemical Co.). GPNMB levels were quantified using the human GPNMB ELISA kit (R&D systems) According to manufacturer's protocol. Briefly, plate was coated with 0.8 μg/ml GPNMB capture antibody overnight. 5 μl of CSF/50 μg protein was added for 2 hours at room temperature. Plate was incubated with biotinylated goat anti mouse antibody for 2 hours at room temperature. Strepavidin-HRP incubation was followed by substrate solution. H₂50₄ was used as a stop solution. Optical density was determined by subtracting reading in 540 nm from reading at 450 nm.

GPNMB quantification in CSF and brain of mice:

Mice were maintained under specific pathogen-free conditions and handled according to protocols approved by the Weizmann Institute Animal Care Committee according to international guidelines.

Gba^{flox/flox}; nestin-Cre mice were used as a model of nGD, in which glucocerebrosidase (GCase) deficiency is restricted to neurons and macroglia (Enquist et al., Proc Natl Acad Sci USA, 104:17483-17488, 2007). Gba^{flox/flox} mice were crossed with Gba^{flox/flox}; nestin-Cre mice to generate Gba^{flox/flox}; nestin-Cre mice (referred to as −/− mice) and Gba^{flox/wt}; nestin-Cre mice (referred to as +/− mice), which served as healthy controls since they do not show any overt pathology (Farfel-Beckeret al., Hum Mol Genet, 20:1375-1386, 2011).

C57BL/6OlaHsd mice were used as another model of nGD, in which the disease is induced by intra-peritoneal (IP) injection with 100 mg/Kg/day conduritol b epoxide (CBD, calbiochem), an irreversible GCase inhibitor.

Mice were anesthetized (100mg/kg Ketamine and 10 mg/Kg xylazine) and CSF was aspired from the Cisterna magna using glass needles. Brain tissues were removed and lysed in ˜6 volumes of Ripa buffer (150 mM sodium chloride, 1.0% Triton X-100, 0.5% sodium deoxycholate, 0.1% SDS, 50 mM Tris, pH 8.0) supplemented with a protease inhibitor mixture (Sigma). Following homogenization, samples were centrifuged at 4500gₐᵥ for 5 min at 4° C., and the supernatant was collected. Protein level was quantified using the BCA protein assay reagent (Pierce Chemical Co.) GPNMB was measured using the mouse GPNMB ELISA kit (R&D systems) According to manufacturer's protocol.

### Example 1

### Identification of Type 3 GD-Associated Proteins

A quantitative global proteomic of cerebrospinal fluid (CSF) samples from four patients with Type 3 GD and 5 age-matched control subjects was performed.

The clinical information of the Type 3 GD patients is presented in Table 1 herein below.

TABLE 1
Clinical information of the Type 3 GD patient participated in the protein profiling

| Sample | Age | | | | |
|---|---|---|---|---|---|
| designation | (Years) | Gender | Genotype | FSIQ* | Comments |
| NO2 | 16 | Female | L444P/L444P | 124 | Only eye movement |
| | | | | | abnormality; probably one |
| | | | | | of the mildest GD Type 3 |
| | | | | | possible |
| NO3 | 13 | Male | L444P/L444P | 45 | Mental retardation |
| NO5 | 8 | Female | L444P/L444P | 74 | Sensory-neural hearing loss |
| N18 | 15 | Male | P122S/P122S | 40 | Progressive myoclonic |
| | | | | | encephalopathy (Gaucher |
| | | | | | type 3A with presumed |
| | | | | | neuronal death) |

*FSIQ—Full Scale IQ.

489 proteins were discovered yet only 7 proteins were found to be elevated by more than 2-fold (P<0.05) and 10 proteins were found to be reduced. Most of the proteins that were found to be elevated are involved in immune response and lipid metabolism.

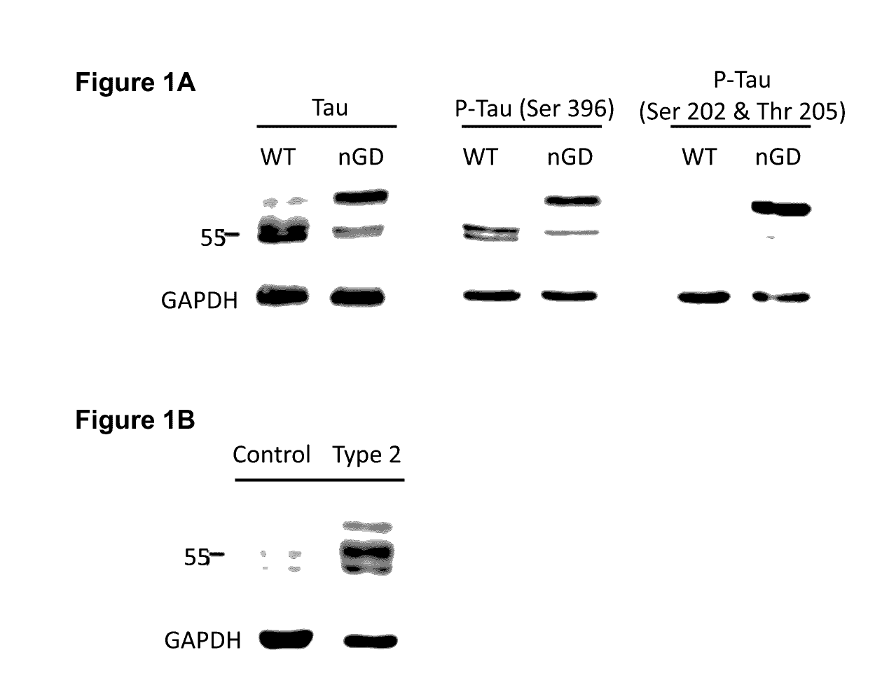

Of the proteins whose levels were reduced, the most significant reduction was for amyloid βA4 (2.3-fold reduction, p<0.05), a key protein in the pathology of Alzheimer's disease. In a subsequent experiment, brains of Gba^{flox/flox}; nestin-Cre mice were tested, and no amyloid formation could be detected at the endstage of Gba^{flox/flox}; nestin-Cre mice. However, hyper-phosphorylated tau, a key player that is linked to amyloid formation in Alzheimer's disease, was detected (FIG. 1A). Hyper-phosphorylated tau was also detected in the brain of a Type 2 GD patient (FIG. 1B). Elevated tau was also recently detected in the brain of type 3 GD patient (Burrow et al., Mol Genet Metab, 114(2):233-41, 2015).

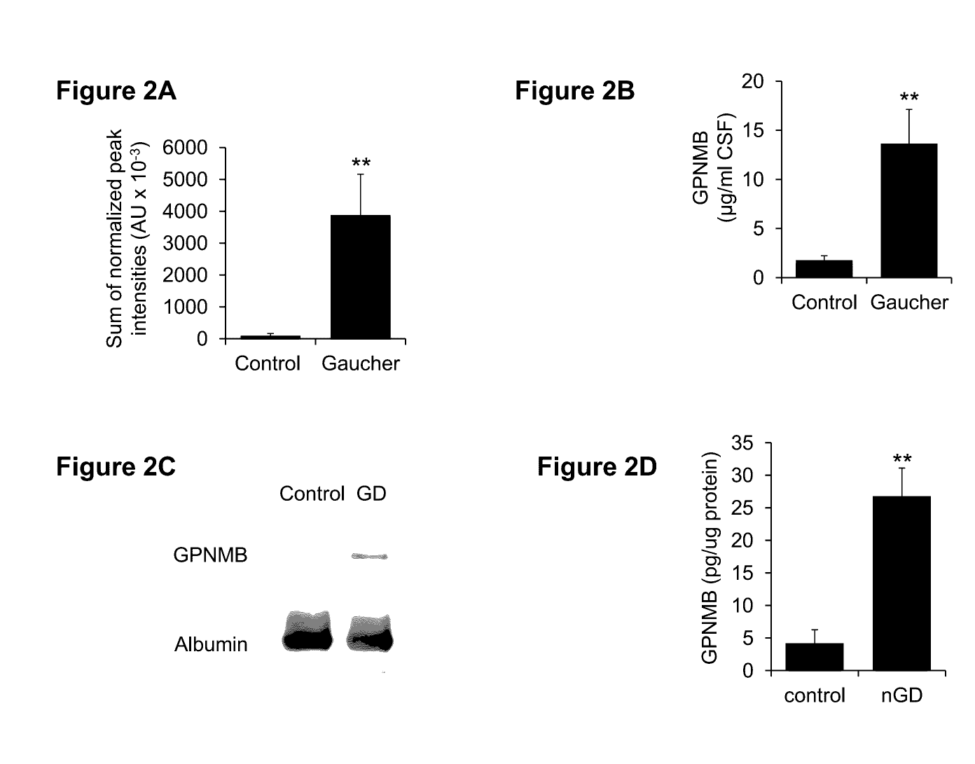

Two proteins were highly elevated in the CSF samples of Type 3 GD patients; Chitotriosidase-1 (CHIT1), a known biomarker for the non neuropathic form of GD (Hollak et al., 1994. ibid) and the glycoprotein non-metastatic B (GPNMB) protein (Table 2, FIG. 2A).

TABLE 2
Up-regulated proteins in CSF samples of Type 3 Gaucher patients

| | | Fold change/ |
|---|---|---|
| Gene symbol | Gene name | p value |
| CHIT1 | Chitotriosidase-1 | 57.7 |
| | | (p < 0.01) |
| GPNMB | Transmembrane glycoprotein NMB | 42.3 |
| | | (p < 0.01) |
| SCTSS | Isoform 2 of Cathepsin | 6.7 |
| | | (p < 0.05) |
| IGKC | Ig kappa chain V-III region GOL | 6.6 |
| | | (p < 0.01) |
| LYZ | Lysozyme C | 6.3 |
| | | (p < 0.01) |
| CFD | Complement factor D | 3.1 |
| | | (p < 0.01) |
| PLD3 | Phospholipase D3 | 2.4 |
| | | (p < 0.01) |

GPNMB level in CSF sample obtained from Type 3 Gaucher's disease was about 42 fold higher compared to its level in CSF samples obtained from healthy subjects.
GPNMB has two isoforms. One having 572 amino acids set forth in SEQ ID NO: 1 and one having 560 amino acids set forth in SEQ ID NO: 2.

| SEQ ID NO: 1 (UniProtKB/Swiss-Prot Q14956.2 | |
|---|---|
| MECLYYFLGF LLLAARLPLD AAKRFHDVLG NERPSAYMRE HNQLNGWSSD ENDWNEKLYP | 60 |
| VWKRGDMRWK NSWKGGRVQA VLTSDSPALV GSNITFAVNL IFPRCQKEDA NGNIVYEKNC | 120 |
| RNEAGLSADP YVYNWTAWSE DSDGENGTGQ SHHNVFPDGK PFPHHPGWRR WNFIYVFHTL | 180 |
| GQYFQKLGRC SVRVSVNTAN VTLGPQLMEV TVYRRHGRAY VPIAQVKDVY VVTDQIPVFV | 240 |
| TMFQKNDRNS SDETFLKDLP IMFDVLIHDP SHFLNYSTIN YKWSFGDNTG LFVSTNHTVN | 300 |
| HTYVLNGTFS LNLTVKAAAP GPCPPPPPPP RPSKPTPSLA TTLKSYDSNT PGPAGDNPLE | 360 |
| LSRIPDENCQ INRYGHFQAT ITIVEGILEV NIIQMTDVLM PVPWPESSLI DFVVTCQGSI | 420 |
| PTEVCTIISD PTCEITQNTV CSPVDVDEMC LLTVRRTFNG SGTYCVNLTL GDDTSLALTS | 480 |
| TLISVPDRDP ASPLRMANSA LISVGCLAIF VTVISLLVYK KHKEYNPIEN SPGNVVRSKG | 540 |
| LSVFLNRAKA VFFPGNQEKD PLLKNQEFKG VS | 572 |

| SEQ ID NO: 2: NP_002501 | |
|---|---|
| MECLYYFLGF LLLAARLPLD AAKRFHDVLG NERPSAYMRE HNQLNGWSSD ENDWNEKLYP | 60 |
| VWKRGDMRWK NSWKGGRVQA VLTSDSPALV GSNITFAVNL IFPRCQKEDA NGNIVYEKNC | 120 |
| RNEAGLSADP YVYNWTAWSE DSDGENGTGQ SHHNVFPDGK PFPHHPGWRR WNFIYVFHIL | 180 |
| GQYFQKLGRC SVRVSVNTAN VTLGPQLMEV TVYRRHGRAY VPIAQVKDVY VVTDQIPVFV | 240 |
| TMFQKNDRNS SDETFLKDLP IMFDVLIHDP SHFLNYSTIN YKWSFGDNTG LFVSTNHTVN | 300 |
| HTYVLNGTFS LNLTVKAAAP GPCPPPPPPP RPSKPTPSLG PAGDNPLELS RIPDENCQIN | 360 |
| RYGHFQATIT IVEGILEVNI IQMTDVLMPV PWPESSLIDF VVTCQGSIPT EVCTIISDPT | 420 |
| CEITQNIVCS PVDVDEMCLL TVRRTFNGSG TYCVNLTLGD DTSLALTSTL ISVPDRDPAS | 490 |
| PLRMANSALI SVGCLAIFVT VISLLVYKKH KEYNPIENSP GNVVRSKGLS VFLNRAKAVF | 540 |
| FPGNQEKDPL LKNQEFKGVS | 560 |

Two peptides from GPNMB were identified in the CSF samples, one peptide comprising the amino acids sequence of positions 219-227 of SEQ ID NOs: 1 and 2 (marked in bold) and another comprising the amino acid sequence of positions 228-246 of these sequences (underlined). Both located within the non-cytosolic (extracellular) domain (Furochi et al., FEBS Lett, 581:5743-5750, 2007) suggesting that GPNMB is cleaved and secreted to the CSF. The non-cytosolic domain of SEQ ID NO: 1 corresponds to positions 1-499. The non-cytosolic domain of SEQ ID NO: 2 corresponds to positions 1-490.

### Example 2

### GPNMB as a Biomarker for nGD

Enzyme-Linked Immunosorbent Assay (ELISA) (FIG. 2B) and Western blot analysis (FIG. 2C) were further performed on the CSF samples to verify that GPNMB levels in CSF of Type 3 Gaucher patients are indicative of the disease compared to its level in the control sample as was found by LC-MS/MS measures. It was indeed verified that in all methods employed, GPNMB levels were elevated in CSF samples of nGD patients compared to the control samples.

Interestingly, a positive correlation between the GPNMB level and disease severity was found, such that higher CSF levels of GPNMB correlated with more severe disease symptoms, along with lower IQ score and lower score in Purdue Pegboard test, assessing eye-hand coordination (Table 3). This suggests that GPNMB in the CSF can be used as a marker of disease severity and for following the progression of CNS pathology in nGD patients.

GPNMB levels were also measured using an ELISA assay in brain samples obtained from Type 2 and Type 3 GD patients versus control brain samples. The results have shown an increased level of GPNMB in the brain samples of the nGD patients compared to the control samples (FIG. 2D). GPNMB levels in the brain samples of Type 2 patients were almost twice as high compared to the levels of Type 3.

TABLE 3
Clinical information of the type 3 GD patients and their
GPNMB levels as measured by LC-MS/MS and ELISA

| | | | | | GPNMB | GPNMB | |
|---|---|---|---|---|---|---|---|
| | | | | Purdue | levels | levels | |
| Sample | Age | | | Pegboard | ELISA | LC/MS | |
| designation | (years) | Gender | FSIQ^{#} | test | (μg/ml) | (AU × 10³)* | Comments |
| N02 | 16 | F | 124 | −1.71 | 6.4 | 792 | Only eye |
| | | | | | | | movement |
| | | | | | | | abnormality; |
| | | | | | | | Mild GD3 |
| N03 | 13 | M | 45 | −3.72 | 19.4 | 4,838 | Mental |
| | | | | | | | retardation |
| NO5 | 8 | F | 74 | −2.93 | 19.5 | 3,932 | Sensoryneural |
| | | | | | | | hearing loss |
| N18 | 15 | M | 40 | −8.73 | 29.6 | 5,958 | Progressive |
| | | | | | | | myoclonic |
| | | | | | | | encephalopathy |
| | | | | | | | (Gaucher type |
| | | | | | | | 3A with |
| | | | | | | | presumed |
| | | | | | | | neuronal death) |

^{#}Full scale IQ
*AU—Arbitrary Units

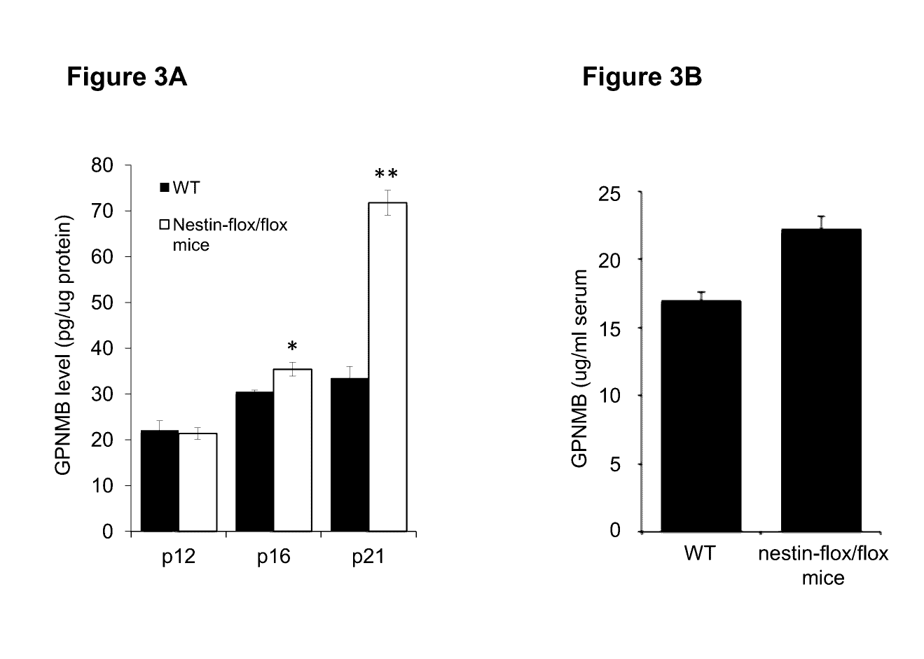

Similar correlation was also observed in a mouse model of GD. Gba^{flox/flox}; nestin-Cre mice, in which GCase deficiency is restricted to neural and glial cells (Enquist, I B et al. 2007. Ibid), showed increasing levels of GPNMB along with disease progression (FIG. 3A). An elevated level in the brain of the Gba^{flox/flox}; nestin-Cre mice, in which GCase deficiency is restricted to neurons and microglia, suggests that the elevation did not originate from the periphery, but rather directly from the brain. A small elevation (1.3-fold) of GPNMB was detected in the serum of the Gba^{flox/flox}; nestin-Cre mice (FIG. 3B), perhaps suggesting some leakage of the CSF into the serum at the late stage in disease progression at which these analysis were performed. Together, these results suggest that GPNMB levels in the CSF could be used as a biomarker to quantify nGD severity.

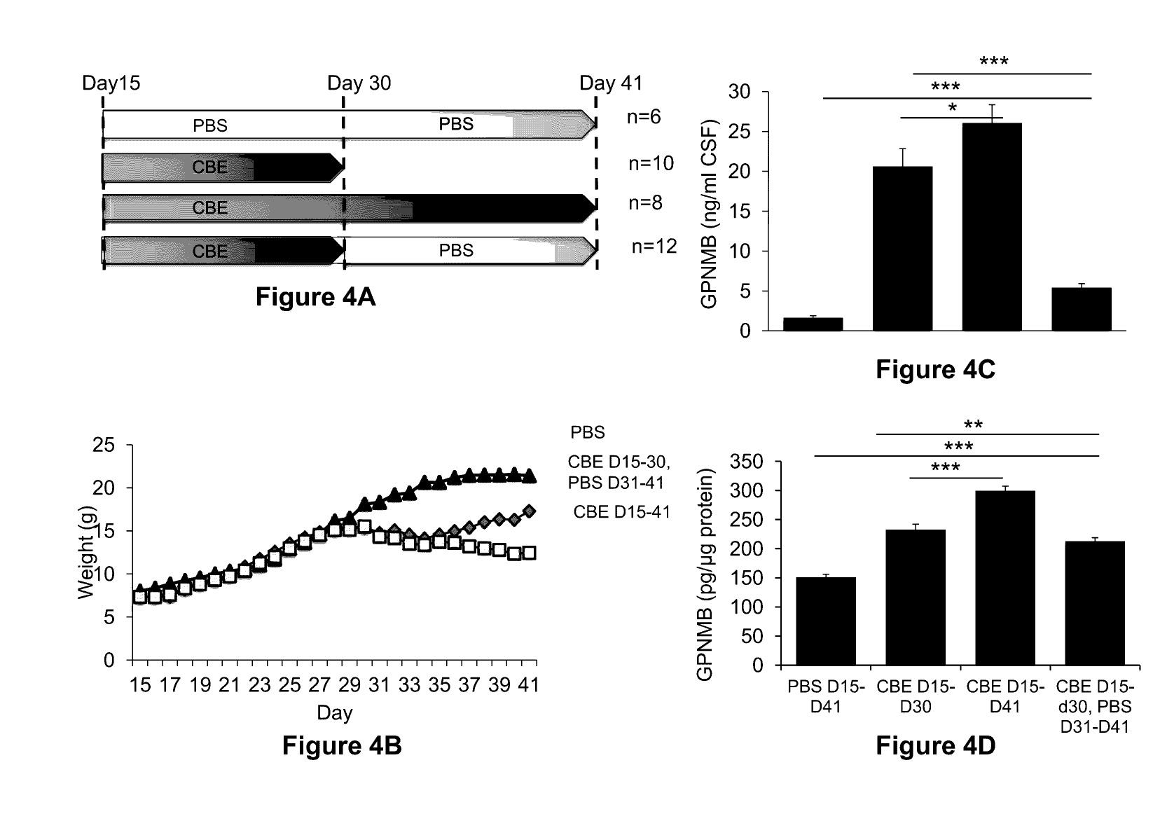

Correlation of CSF levels of GPNMB and nGD symptoms was further verified using an additional mouse model of GD, where mice are induced for GD by injections of conduritol b epoxide (CBE), a GCase inhibitor. 100 mg/Kg/day of CBE were daily injected into mice starting on day 15 post-natal. On day 30 the mice were divided into three groups: “CBE D15-30” in which CBE injections were ceased on day 30, “CBE D15-30; PBS D31-41” in which CBE injections were ceased on day 30 and the mice continued to receive phosphate buffered saline (PBS) injections until day 41, and “CBE D15-41”, in which CBE injections were continued to day 41. An additional group of mice was injected with PBS during the entire experiment (“PBS”) and served as a control (FIG. 4A). Mice body weight, a simple indicator of disease progression, was measured daily during the experiment and the results are shown in FIG. 4B. The figure shows that the body weight began to decrease ˜10-12 days after beginning CBE injections and continued to decrease until day 41 in the CBE D15-41 group. However, mice in which CBE injections ceased on day 31 began to gain weight. This assay demonstrates inducing early and late stage of GD, as well as a “recovering” stage for the groups of mice where CBE injections were ceased.

CSF was aspirated from the Cisterna magna of the mice and GPNMB levels were measured in both CSF (FIG. 4C) and brain homogenates (FIG. 4D) by ELISA. GPNMB levels in the CSF and brain correlated with changes in body weight and with neurological signs of nGD: marked elevation of GPNMB was detected in the brain and CSF of CBE treated mice. Remarkably, GPNMB levels rapidly declined during the recovery period. More specifically, GPNMB levels in the CSF and brain increased upon injection of CBE from day 15-30 and to a higher extent with injection until day 41. Upon cessation of CBE injection on day 30, a significantly lower level of CSF/brain GPNMB was detected on day 41 than in mice that were continuously treated with CBE until day 41.

To confirm the purity of the mouse CSF samples used in this study, the percent of polynuclear cells in CSF was analyzed by FACS. Polynuclear cells comprised ˜0.55% of the cells in the CSF (n=5), whereas blood samples contained 10% polynuclear cells, indicating the high purity of the CSF samples. Together, these results suggest that analysis of GPNMB levels in the CSF could provide a means to determine disease progression as well as treatment efficacy.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

What is claimed is:

1. A method for assessing the responsiveness of a subject diagnosed with neuronopathic Gaucher's disease (nGD) to a treatment, the method comprising:
(a) measuring a level of GPNMB or a fragment thereof in a first cerebrospinal fluid (CSF) sample obtained from the subject;
(b) administering the treatment to said subject;
(c) measuring a level of GPNMB or the fragment thereof in a second CSF sample obtained from said subject at a selected time period after the treatment was administered; and
(d) calculating a ratio of the level of GPNMB or the fragment thereof in the first CSF sample to the level of GPNMB or the fragment thereof in the second CSF sample;
wherein a ratio of greater than 1 identifies said subject as responsive to said treatment.

2. The method of claim 1, wherein GPNMB has an amino acid sequence selected from the group consisting of SEQ ID NO: 1 and SEQ ID NO: 2.

3. The method of claim 1, wherein the fragment of GPNMB is derived from the extracellular domain of GPNMB.

4. The method of claim 1, wherein measuring a level of GPNMB or a fragment thereof is performed using an immunologic technique.

5. The method of claim 4, wherein the immunologic technique is selected from the group consisting of fluorescence immunoassay (FIA) method, an enzyme immunoassay (EIA) method, a radioimmunoas say (RIA) method, a Western blotting method and slot blot.

6. The method of claim 1, wherein the method further comprises repeating steps (c) and (d) at least once, wherein a third or more samples are obtained in step (c) according to the number of repeats.

7. A method for diagnosing neuronopathic Gaucher's disease (nGD) in a subject, the method comprising:
(a) receiving a cerebrospinal fluid (CSF) sample from a subject suspected of having nGD;
(b) determining a level of GPNMB or a fragment thereof in the CSF sample using a quantitative assay;
(c) comparing the level of GPNMB or the fragment thereof in the CSF sample to a reference value representing a normal level of GPNMB in the CSF; and
(d) diagnosing the subject as having nGD if the level of GPNMB or the fragment thereof in the CSF sample is above the reference value.

8. The method of claim 7, wherein GPNMB has an amino acid sequence selected from the group consisting of SEQ ID NO: 1 and SEQ ID NO: 2.

9. The method of claim 7, wherein the fragment of GPNMB is derived from the extracellular domain of GPNMB.

10. The method of claim 7, wherein the quantitative assay is an immunoassay.

11. The method of claim 10, wherein the immunoassay is selected from the group consisting of fluorescence immunoassay (FIA) method, an enzyme immunoassay (EIA) method, a radioimmunoassay (RIA) method, a Western blotting method and slot blot.

12. The method of claim 7, further comprising administering treatment to the subject.

13. The method of claim 7, further comprising determining disease severity.

14. The method of claim 13, wherein determining disease severity comprises providing a plurality of reference values, each representing a level of GPNMB in the CSF that is characteristic of a degree of disease severity; comparing the level of GPNMB or the fragment thereof measured in the CSF sample from the subject to each reference value; and determining the degree of disease severity in the subject based on the reference value which correlates best with the level of GPNMB or the fragment thereof in the CSF sample from the subject.

15. The method of claim 14, wherein each reference value represents a range of levels of GPNMB in the CSF that is characteristic of a degree of disease severity.

16. A method for monitoring the progression of neuronopathic Gaucher's disease (nGD) in a subject diagnosed with nGD, the method comprising:
(a) measuring a first level of GPNMB or a fragment thereof in a first cerebrospinal fluid (CSF) sample taken from a subject diagnosed with nGD;
(b) measuring a second level of GPNMB or the fragment thereof in a second CSF sample taken from the subject after a predetermined period of time;
(c) comparing the first and second levels of GPNMB or the fragment thereof; and
(d) determining disease progression if the second level of GPNMB or the fragment thereof is significantly increased compared to first level of GPNMB or the fragment thereof.

17. The method of claim 16, wherein the first level of GPNMB or the fragment thereof is measured before treatment and the second level of GPNMB or the fragment thereof is measured after treatment.

18. The method of claim 17, further comprising determining improvement of the disease if the second level of GPNMB or the fragment thereof measured after treatment is significantly reduced compared to the first level of GPNMB or the fragment thereof measured before treatment.
